(19) United States
(12) Patent Application Publication
(10) Pub. No.: US 2006/0110370 A1
(43) Pub. Date: May 25, 2006

(54) Treatments for reduction of cytotoxicity and viral contamination of implantable medical devices

(76) Inventors: Chandrashenkhar P. Pathak, Phoenix, AZ (US); Mark A. Moore, Chesapeake, VA (US); Charles Simpson, Scottsdale, AZ (US); Thomas Simpson, Pflugerville, TX (US); Sallie McLaughlin, Volente, TX (US)

Correspondence Address: WILLIAMS, MORGAN & AMERSON, 10333 RICHMOND, SUITE 1100, HOUSTON, TX, 77042, US

(21) Appl. No.: 10/996,156

(22) Filed: Nov. 23, 2004

(51) Int. Cl. A61K 35/12; A01N 1/02

(52) U.S. Cl. 424/93.7; 435/5; 435/1.1

(57) ABSTRACT

A method for treating biomaterial is provided in which a biological tissue, typically after being cross-linked, is contacted with an anticalcification treatment solution under condition effective to render the biomaterial resistant to in vivo calcification upon implantation in a host animal. The anticalcification treatment solutions comprise higher alcohol solutions, a polyol solutions and/or a polar aprotic organic solvent solutions. Methods of reducing cytotoxicity to host tissue of bioprostheses that comprise fixed animal tissues, and treatments to reduce viral contamination of implantable medical devices are disclosed herein.

### 1.0 BACKGROUND OF THE INVENTION

1.1 Filed of the Invention

The present invention relates generally to the field of medical devices for implantation into humans. More particularly, the present invention concerns methods for processing biological materials for use as bioprosthetic implantable devices. The present invention also concerns treatments to reduce cytotoxicity to host tissue of bioprostheses that comprise fixed animal tissues, and treatments to reduce viral contamination of implantable medical devices.

1.2 Description of the Related Art

Bioprostheses are devices derived from processed biological tissues to be used for implantation into humans. The development of such devices originated as an attempt to circumvent some of the clinical complications associated with the early development of the mechanical heart valve, and has since resulted in a rapid proliferation of bioprosthetic devices for a variety of applications. Examples of some of the bioprostheses currently used or under development include heart valves, vascular grafts, biohybrid vascular grafts, ligament substitutes, pericardial patches, and others.

Processing of biological tissues that are to be used in preparing bioprostheses can include some or all of the steps of: cleaning and preserving the tissue, crosslinking or fixing the tissue, anticalcification treatment, assembly of a finished bioprosthesis comprising the tissue (e.g., sewing techniques, among others), and sterilizing and packaging the tissue or a bioprosthesis that comprises it.

The primary component of the biological tissues used to fabricate bioprostheses is collagen, a generic term for a family of related extracellular proteins. Collagen molecules consist of three chains of poly(amino acids) arranged in a trihelical configuration ending in non-helical carboxyl and amino termini. These collagen molecules assemble to form microfibrils, which in turn assemble into fibrils, resulting in collagen fibers. The amino acids which make up the collagen molecules contain side groups, including amine (NH₂), carboxylic acid (COOH) and hydroxyl (OH) groups, in addition to the amide bonds of the polymer backbone, all of which represent sites for potential chemical reaction on these molecules.

Because collagenous tissues degrade rapidly upon implantation into a host recipient, it is necessary to stabilize the tissue if it is to be used for clinical applications. Chemical stabilization by tissue cross-linking, also known as tissue fixation, has been achieved using a variety of compounds. Most typically, chemical fixation has employed polyfunctional molecules having two or more reactive groups capable of forming irreversible and stable intramolecular and intermolecular chemical bonds with the reactive amino acid side groups present on the collagen molecules. The most widely used of these polyfunctional molecules is the five carbon molecule, glutaraldehyde, which has an aldehyde at each end of a linear aliphatic chain. The aldehyde groups of glutaraldehyde and other like molecules react under physiological conditions with the primary amine groups of collagen molecules to cross-link the material. Glutaraldehyde cross-linked tissue produced in this way exhibits increased resistance to enzymatic degradation, reduced immunogenicity, and increased stability.

Despite its widespread use, there are certain disadvantages associated with tissue cross-linking with polyfunctional aldehydes and other chemical cross-linking agents. For example, upon implantation, aldehyde fixed tissue is susceptible to the formation of degenerative calcific deposits. Pathologic calcification, e.g., the undesirable deposition of calcium phosphate mineral salts in an implanted tissue, may represent the predominant cause of failure of glutaraldehyde-fixed bioprosthetic devices (Golomb et al., 1987; Levy et al., 1986; Thubrikar et al., 1983; Girardot et al., 1995). The mechanism for pathological calcification of implanted tissue is not fully understood, but may be due to host factors, implant factors, and/or extraneous factors, such as mechanical stress. Additionally, there is some evidence to suggest that deposits of calcium may be related to devitalized cells, and, in particular, to cell membranes in which the calcium pumps (Ca⁺²—Mg⁺² ATPase) responsible for maintaining low intracellular calcium levels are no longer functioning or are malfunctioning.

Detergent pretreatment with non-covalently linked detergents, such as sodium dodecyl sulfate (SDS), or covalently bound detergents, such as amino oleic acid, have been reported to reduce calcification of materials exposed to circulating blood (Gott et al., 1992). However, detergents can adversely affect tissue structure and/or properties, resulting in a diminution of the collagen denaturation temperature, or shrink temperature, which is an important measure of material strength, durability, and integrity. Moreover, use of detergents can result in local toxicity.

In another approach, U.S. Pat. No. 5,746,775 describes the treatment of glutaraldehyde pretreated tissue with lower alcohols (i.e., C₁-C₃ alcohols), in which the lower alcohol is present at greater than 50% by volume in an alcohol treatment solution. The method is reported to be useful in preparing tissue for implantation into a living being. However, if a bioprosthesis comprising porcine, bovine, or human aortic root tissue is stored in such a solution, significant swelling (e.g., blistering) of the spongiosa of the aortic root can, in certain cases, result, and the blistered aortic root tissue can be unacceptable for implantation.

Certain biological tissues for use in implants that are commercially available are stored in buffered polyfunctional compounds, such as glutaraldehyde. This is particularly the case when the biological tissue has been fixed using a polyfunctional molecule like glutaraldehyde. However, polyfunctional compounds, such as glutaraldehyde, can often be difficult to remove from a biological tissue that has been stored in a solution that comprises the compound, and as a result biological tissue (e.g., heart valve) can be cytotoxic to host tissue upon implantation. Therefore, there is interest in finding methods to reduce the cytotoxicity of tissues that are fixed with polyfunctional compounds like glutaraldehyde.

During the preparation and manufacture of implantable medical devices, it is possible for the devices to become contaminated with one or more viruses. For example, an implantable medical device may be prepared using a tissue from a virally infected animal, or the device can become contaminated at some point during processing before implantation. Viruses present in/on an implantable medical device can in certain cases cause an unfavorable inflammatory response/infection in the host animal upon implantation of the device. Therefore, methods for reducing or eliminating viral contamination that might be present on an implantable medical device before implantation are desirable.

Despite previous attempts at providing biomaterials having resistance to calcification, there remains a need for alternative anticalcification approaches with improved efficacy and ease of use. There is, thus, a need for an effective method of imparting long-term anticalcification properties to bioprosthetic materials, e.g., tissues, that is not accompanied by deleterious effects and that incorporate anticalcification agents and/or treatments into existing protocols for the preparation of clinical-grade biomaterials. The present invention is directed to overcoming or at least reducing the effects of one or more of the problems set forth above.

### 2.0 SUMMARY OF THE INVENTION

According to one aspect of the present invention, there is provided a method for treating a biomaterial comprising contacting a biomaterial, such as a cross-linked animal tissue, with an anticalcification treatment solution. The anticalcification treatment solutions of this aspect of the invention include solutions comprised higher alcohols or polyols and polar aprotic organic solvents. The anticalcification treatment solutions are contacted with the biomaterial under conditions effective to reduce pathologic calcification of the biomaterial following implantation into a mammalian host. As illustrated herein, this reduction in calcification can be monitored, for example, by evaluating the calcium content of an implanted biomaterial treated with an anticalcification treatment solution of the invention compared with an implanted biomaterial not so treated. Preferably, this reduction in calcification will be greater than 50%, more preferably greater than 75%, and most preferably greater than 90%, compared with an implanted, untreated biomaterial.The higher alcohol or polyol used in formulating the anticalcification treatment solution may be a linear or branched C₄-C₃₆ alcohol or polyol. In certain preferred embodiments of the invention, the higher alcohol or polyol will be selected from a C₆-C₁₈ alcohol or polyol, preferably from a C₇-C₉ alcohol or polyol. Typically, the higher alcohol or polyol comprises less than about 50% by volume of said anticalcification treatment solution. In some instances, however, it will be desired to use an anticalcification treatment solution wherein the higher alcohol or polyol comprises less than about 25% by volume of said anticalcification treatment solution, or even less than about 10% by volume of said anticalcification treatment solution. The anticalcification treatment solution of the present invention may further comprise at least one organic solvent selected from, for example, C₁-C₃ alcohols. Moreover, the anticalcification treatment solution can also comprise water or an aqueous solvent.

Polar aprotic organic solvents useful in formulating the anticalcification treatment solutions of the present invention will preferably have dielectric constants greater than about 20, more preferably greater than about 30, and they will possess some degree of water solubility. Polar aprotic organic solvents useful in this aspect of the invention include, for example, N-alkyl pyrolidinones and N-alkyl amides, in which the alkyl group or groups comprise branched or linear alkyl chains having from about 1 to 10 carbon atoms. Illustrative solvents of this class include N-methyl pyrolidinone, N,N-dimethylacetamide, N,N-dimethylformamide, N,N-dimethylpropionamide, and the like.

In a further aspect of the present invention, there is provided a method for treating an aldehyde cross-linked animal tissue by forming an anticalcification treatment solution comprised of at least one organic solvent and from about 0.1% to about 25% by volume of a C₆-C₁₈ alcohol or polyol, and contacting the anticalcification treatment solution with the aldehyde cross-linked biomaterial under conditions effective to reduce pathologic calcification of the biomaterial following implantation into a mammalian host. As described above, an anticalcification treatment solution of the invention may contain one or more organic solvents and may further comprise water or a compatible aqueous solvent system. In one illustrative embodiment of this aspect of the invention, an organic solvent is present at about 35% to about 49% by volume of said anticalcification treatment solution, the remainder being comprised of said water or aqueous solvent. In this embodiment, it is preferred that the water or aqueous solvent is present at greater than about 50% by volume of said anticalcification treatment solution.

In yet a further aspect of the present invention, there is provided a method for treating an aldehyde cross-linked mammalian tissue by providing an anticalcification treatment solution comprised of about 0.1% to about 25% by volume of a C₆-C₁₈ alcohol or polyol, about 25% to about 99% by volume of an organic solvent selected from a C₁-C₃ alcohol, the remaining volume, if any, being comprised of water or an aqueous solvent; and contacting the anticalcification treatment solution with an aldehyde cross-linked biomaterial for a duration effective to reduce pathologic calcification of the biomaterial following implantation into a mammalian host. One illustrative embodiment of this aspect of the invention employs an organic solvent that is present at about 35% to about 45% by volume of the anticalcification treatment solution and a higher alcohol or polyol that is present at about 1% to about 10% by volume of the anticalcification treatment solution.

In another aspect of this invention, a method is provided for treating a biomaterial, comprising contacting an aldehyde-cross-linked biomaterial with an anticalcification treatment solution comprised of N-methyl pyrolidinone, N,N-dimethylacetamide, N,N-dimethylformamide and/or N,N-dimethylpropionamide under conditions effective to reduce pathologic calcification of the biomaterial following implantation into a mammalian host.

In another aspect of this invention, a method is provided for treating a biomaterial, preferably a cross-linked animal tissue, by contacting a biomaterial with an anticalcification treatment solution at a temperature between about 30° and 60° C. for a duration and under conditions effective to reduce pathologic calcification of the biomaterial following implantation into a mammalian host. The anticalcification treatment solutions comprise between about 10% and about 50% by volume, preferably between about 25% and 50% by volume, of a C₁-C₃ alcohol, such as methanol, ethanol, propanol, or isopropanol, the remaining volume being comprised of water or an aqueous buffer, such as HEPES.

Certain embodiments of the present invention are directed to methods of treating a bioprosthesis to reduce its cytotoxicity and to bioprostheses prepared using such methods. The bioprosthesis comprises an animal tissue that has been fixed with a chemical cross-linking agent, and the bioprosthesis is cytotoxic to host tissue upon implantation into an animal (e.g., a human). The bioprosthesis can comprise, for example, human, bovine, or porcine tissue, and the chemical cross-linking agent can comprise, for example, an aldehyde. The bioprosthesis is contacted with an aqueous composition comprising at least one salt, and a C₁-C₃ alcohol. The bioprosthesis is less cytotoxic after being contacted with the aqueous composition than before being contacted with the aqueous composition. In other words contact with the aqueous composition is effective to reduce the cytotoxicity of the bioprosthesis. In certain embodiments the aqueous composition further comprises a C₄-C₃₆ alcohol. Preferably the aqueous composition comprises between about 0.1 and 10% by volume of a C₆-C₁₈ alcohol, between about 15 and 25% by volume of a Cₗ-C₃ alcohol, and between about 65 and 85% by volume of an aqueous salt solution. The aqueous salt solution can, optionally comprise a chemical buffer.

Other embodiments of the present invention are directed to methods of treating an implantable medical device to reduce its viral contamination, and to implantable medical devices prepared using such methods. In certain embodiments the implantable medical device comprises, for example, a human, bovine, or porcine animal tissue, and the animal tissue can be fixed in certain embodiments. The implantable medical device comprises at least one viral contaminant, and is contacted with an aqueous composition comprising at least one salt, a C₄-C₃₆ alcohol, and a C₁-C₃ alcohol. The implantable medical device comprises less viral contaminant after being contacted with the aqueous composition than before being contacted with the aqueous composition. In certain embodiments the amount of viral contaminant is reduced by at least about one log by contacting the implantable medical device with the aqueous composition. It is preferred that the aqueous composition comprises between about 0.1 and 10% by volume of a C₆-C₁₈ alcohol, between about 15 and 25% by volume of a C₁-C₃ alcohol, and between about 65 and 85% by volume of an aqueous salt solution. More specifically, the present invention can make a tissue-based bioprostheses, such as heart valve bioprosthesis, less cytotoxic and/or have a reduced viral load without affecting the valve performance, such as durability, resistance to calcification, and other fluid flow characteristics.

### 3.0 DESCRIPTION OF ILLUSTRATIVE EMBODIMENTS

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

Implantable medical devices used in methods of the present invention can be any known in the art. Exemplary implantable medical devices are heart valves, vascular grafts, biohybrid vascular grafts, ligament substitutes, and pericardial patches, among others. While in certain embodiments the implantable medical device does not necessarily comprise an animal tissue, in certain embodiments the implantable medical device can be a bioprosthesis that comprises an animal tissue. The term “biomaterial” is used herein to refer generally to collagen-containing, biologically-derived materials. For example, various types of implantable biological tissues derived from numerous animal sources and parts of the anatomy can be used as biomaterials in accordance with this invention. The tissue can be derived, for example, from animal sources such as human, bovine, porcine, equine, sheep, kangaroo, rabbit, and others. Preferably the tissue is a human, bovine, or porcine tissue. Illustrative examples of animal tissues used in accordance with the present invention include, without limitation, heart valves, particularly porcine or bovine heart valves; aortic roots, walls, and/or leaflets; pericardium; connective tissue-derived materials such as dura mater; homograft tissues such as aortic homografts and saphenous bypass grafts; tendons; ligaments; skin patches; arteries; veins; and the like, Of course, other biologically-derived materials that are known as being suitable for in-dwelling uses in the body of a living being are also within the contemplation of the invention. In certain embodiments a sewing cuff is coupled to the implantable medical device, and in some instances the sewing cuff is attached to a biomaterial component. For some applications, it may be desired to manipulate a biomaterial in some manner so as to provide it in a particular form or shape, for example using metallic stents prior to the treatments described herein. In this way, the biomaterial may be cross-linked and/or alcohol treated in the particular three-dimensional geometric configuration of the bioprosthesis to be implanted.

Implantable medical devices, such as bioprostheses, can optionally be rinsed before being implanted in a patient (e.g., animal) or before or after undergoing a treatment, as described below. During rinsing, the implantable medical device is preferably shaken, or intermittently stirred, to ensure even distribution of the rinse fluid. Exemplary rinsing fluids include physiologically suitable fluids, such as water, and solutions such as saline, phosphate buffered saline (PBS), HEPES buffered saline, ringers lactate (pH 7.4), sodium bicarbonate (pH 7.4), tris buffer (pH 7.4), imidazole (pH 7.4), and the like. Preferably the implantable medical device is rinsed for between about 1 minute and 100 hours. Preferably the rinsing is at a temperature between about 25 and 40 degrees Celsius.

In certain embodiments of the present invention, an animal tissue has not been chemically fixed. However, typically, the biomaterial treated according to this invention is comprised of a biomaterial that has been fixed/cross-linked by treatment with one or more chemical cross-linking agents or other treatments that effect cross-linking (e.g., photooxidation). These can include, for example, treatments with polyfunctional aldehydes, polyfunctional epoxides, photoxidation and/or any other cross-linking agents or treatments that promote reactions between carboxylic acid and amine groups, such as N-(3-Dimethylaminopropyl)-N′-ethylcarbodiimide hydrochloride (EDC). Of course, the anticalcification treatments of this invention are preferably used in conjunction with cross-linking agents or treatments that increase the propensity of a biomaterial to calcify following implantation into a living host. Similarly cytotoxicity reducing treatments of this invention are preferably used in conjunction with bioprostheses comprising a chemically cross-linked animal tissue. In certain embodiments the viral contamination reducing treatments of this can be used to treat implantable medical devices comprising a cross-linked animal tissue (e.g., by chemical fixation or photooxidation). In other embodiments the implantable medical device treated with an aqueous composition that reduces viral contamination does not comprise a biomaterial (e.g., unfixed or fixed).

In one embodiment of the invention, the biomaterial is one that has been cross-linked by treatment with a monofunctional aldehyde, a polyfunctional aldehyde, or some combination thereof. A “monofunctional aldehyde” refers to a molecule containing a single aldehyde functionality, such as formaldehyde, while “polyfunctional aldehyde” refers to a molecule that contains two or more aldehyde functionalities. The other constituents present on the monofunctional or polyfunctional aldehyde are not critical provided they do not adversely effect the ability of the aldehyde groups to be collagen-reactive, and thereby capable of producing cross-linked biological tissues. Examples of monofunctional and polyfunctional aldehydes commonly used in biomaterial fixation methods for producing cross-linked biomaterials include aldehyde compounds that contain an aliphatic component comprising a linear or branched, saturated or unsaturated aliphatic chain having from about 2 to about 36 carbon atoms. Most preferably, cross-linking processes employ the use of a polyfunctional aldehyde having from 2 to about 10 carbon atoms, such as the linear five-carbon alkyl dialdehyde, glutaraldehyde.

As used herein, the terms “aldehyde fixed biomaterial” or “aldehyde cross-linked biomaterial” refers to biomaterial that has been treated with one or more monofunctional and/or polyfunctional aldehyde compounds. The techniques and conditions for treating biomaterials with aldehyde-containing cross-linking agents are well known and are readily available to the skilled individual in the art (for example, see Zilla et al.). In these processes, a biomaterial is typically contacted with an aldehyde solution for a duration and under conditions effective to result in the desired degree of cross-linking of collagen and other cellular proteins within the tissue. Procedures for monitoring the progress and/or completion of the cross-linking reaction are also well known. For example, the degree of cross-linking of a treated tissue can be monitored by evaluating its shrinkage temperature and/or the quantity of extractable protein present in the material.

The skilled individual in this art will recognize that the duration of the cross-linking reaction according to this invention is not critical so long as the biomaterial and the cross-linking agent remain in contact for a time sufficient to allow cross-linking to occur. Time of treatment will of course vary depending on the type of biomaterial being treated, the particular aldehyde used and/or the concentration of the aldehyde in the cross-linking solution. Typically, the length of the reaction will be from about one minute to several days. However, the time of treatment should not be so long as to adversely effect the cross-linked biomaterial. Cross-linking times of several days or more are not uncommon. However, the biomaterial can also be treated for shorter periods as well, e.g., from about one minute to about twelve hours, or for about one hour to about six hours, provided the desired degree of cross-linking is achieved.

The reaction temperatures and/or pressures employed in a typical cross-linking reaction are not critical and can include essentially any conditions that are effective for allowing the cross-linking reaction to occur while not adversely compromising the progression of the reaction or the integrity of the biomaterial being treated. Identification of optimal temperature and pressure conditions for a particular implementation of the present invention can be readily determined by the skilled individual in this art. Generally, the cross-linking reaction can be carried out at an ambient temperature, or at any other convenient temperature that does not substantially exceed the tissue denaturation temperature of about 62° C. Thus, reaction temperatures may be selected from a temperature range from about 0° C. to about 60° C., preferably from about 20° C. to about 50° C. Although the pressure for a typical reaction generally ranges from about 2 mm Hg to about 6 mm Hg, suitable pressures may be as high as 100 mm Hg, or more, if desired. Certain biomaterials that comprise chemical cross-linking agents can be cytotoxic upon being implanted.

In certain embodiments a biomaterial optionally can be contacted with an anticalcification treatment solution, after the biomaterial is cross linked in the manner described above, and the tissue is optionally washed/rinsed. The anticalcification treatment solutions of the present invention include solutions comprised of higher alcohols, polyols (i.e., organic molecules containing two more alcohol functionalities), polar aprotic solvents, such as N-methyl pyrolidinone, and solutions comprised of less than about 50% by volume of one or more lower (C₁-C₃) alcohols.

Therefore, according to one embodiment of the present invention, the anticalcification treatment solutions is comprised of one or more higher alcohols or polyols (e.g., a C₄ to C₃₆ alcohol or polyol). The higher alcohol or polyol will typically be an aliphatic linear or branched alcohol or polyol, and may contain additional chemical moieties or substituents provided they do not unacceptably interfere with the anticalcification effects described herein. In one illustrative embodiment of the invention, the higher alcohols used to formulate and anticalcification treatment solution are primary, secondary or tertiary alcohols selected from linear or branched C₆-C₁₈ aliphatic alcohols, such as hexanol, heptanol, octanol, nonanol, etc., or linear or branched C₆-C₁₈ polyols selected from 1,2-octanediol (also sometimes referred to as 1,2-dihydroxyoctane), 1,8-octanediol, 1,10-decanol, 1,10-dodecanol, 1,2-dihydroxydecane and 1,2-dihydroxydodecane.

In certain illustrative embodiments of the invention, the higher alcohols or polyols are present at less than about 50%, less than about 25%, or less than about 10%, by volume of the anticalcification treatment solution, the remainder being comprised of an organic solvent. Thus, in addition to the higher alcohols and polyols described above, the anticalcification treatment solution of the present invention may further contain one or more organic solvents. The organic solvents used in accordance with the present invention are preferably selected from those that do not have deleterious effects on the tissue being treated or on the anticalcification effects achieved by use of the anticalcification treatment solution. The organic solvents should be capable of adequately dissolving the higher alcohol or polyol to form a homogeneous anticalcification treatment solution. Organic solvents that can improve, enhance, or otherwise facilitate the anticalcification effects of the higher alcohols or polyols of this invention are, of course, particularly preferred. Organic solvents useful in accordance with this embodiment include lower alcohols (e.g., C1-C3 alcohols), acetone, ethyl acetate, ethyl lactate, 1,4-butanediol, polyethylene glycol, and the like.

Anticalcification treatment solutions according to certain embodiments of the invention comprise one or more higher alcohols and/or polyols in a preferably homogeneous mixture with one or more organic solvents. For example, particularly illustrative alcohol treatment solutions comprise from about 0.1% to about 25% by volume of one or more higher alcohols or polyols, with substantially all of the remainder of said solution being comprised of organic solvent. Additional illustrative anticalcification treatment solutions comprise from about 0.1% to about 10% by volume of one or more higher alcohols or polyols, with substantially all of the remainder of the solution being comprised of organic solvent.

Alternatively, the one or more higher alcohols or polyols of the anticalcification treatment solution may be formulated in an aqueous solvent system, e.g., with water or with any of a variety of aqueous buffer systems, or may be formulated in a mixture of an aqueous solvent system and one or more organic solvents. Some higher alcohols and polyols may exhibit poor solubility in aqueous based systems, but have greater solubility in many organic solvents. Thus, in embodiments which employ an aqueous based solvent systems, it will in some instances be preferred that one or more organic solvents is also employed in an amount at least sufficient to dissolve the higher alcohol or polyol to provide a homogeneous, i.e., substantially single-phase, anticalcification treatment solution.

Therefore, in additional embodiments of the invention, anticalcification treatment solutions are comprised of about 0.1% to about 25% by volume of one or more higher alcohols or polyols, about 25% to about 49% by volume of one or more organic solvents, with substantially all of the remainder of said solution being water or an aqueous based solution. Further embodiments of the invention provide anticalcification treatment solution comprised of about 0.1% to about 10% by volume of one or more higher alcohols or polyols, about 35% to about 45% by volume of one or more organic solvents, with substantially all of the remainder of said solution being water or an aqueous based solution.

In another embodiment of this invention, the anticalcification treatment solution is comprised of one or more polar aprotic solvents. Such solvents can include, for example, N-alkyl pyrolidinones and N-alkyl amides, in which the alkyl group or groups comprise linear or branched alkyl chains having from about 1 to 10 carbon atoms. Illustrative solvents of this type include N-methyl pyrolidinone, N,N-dimethylacetamide, N,N-dimethylformamide, N,N-dimethylpropionamide, and the like. Particularly preferred polar aprotic solvents include those having some degree of water solubility and/or those with high dielectric constants, for example having dielectric constants greater than about 20, preferably greater than about 30.

In yet another embodiment of the invention, lower (C₁-C₃) alcohol treatment solutions, comprising less than 50% by volume of the lower alcohol, preferably between about 25% and 50%, are also suitable as anticalcification treatment solutions. Whereas prior anticalcification treatment attempts using lower alcohol solutions such as these have been unsuccessful, it has now been found that significant anticalcification effects can indeed be achieved by contacting a biomaterial with a lower alcohol treatment solution at a temperature in the range of about 30° C. to about 60° C., preferably between about 35° C. and 45° C. These treatment temperatures improve the efficacy of the anticalcification treatment solutions of this embodiment, possibly by facilitating the diffusion and penetration of the lower alcohols into the biomaterial. Preferably, the treatment according to this embodiment is accompanied by agitation of the anticalcification treatment solution while it is in contact with the biomaterial.

Cross-linked biomaterial is contacted with, or otherwise exposed to, an anticalcification treatment solution of the present invention for a period of time sufficient to render the biomaterial more resistant to in vivo pathologic calcification than a biomaterial not treated with the anticalcification treatment solution. The length of exposure in the embodiments described herein is illustrative only and can be varied by those of skill in the art while achieving a desired result. For embodiments of the invention wherein the biomaterial is immersed or soaked in a liquid anticalcification treatment solution, the exposure time will typically be in the range of about 1 hour to about 96 hours. For some biomaterials, excessive exposure to the anticalcification treatment solution may result in a decrease in the anticalcification effects, or may necessitate rehydration of the tissue.

The treatment procedure can be carried out at or near room temperature (e.g., about 25° C.) if desired. However, any temperature of convenience that is not deleterious to the biomaterial, for example about 4° C. to about 60° C., may also be used. As discussed above, it may indeed be desired and/or necessary in some embodiments to use an incubation temperature greater than room temperature in order to improve the efficacy of the treatment process, for example by increasing the rate and/or degree of diffusion and penetration of the anticalcification solutions into the biomaterial.

The biomaterial will typically be treated by contact with a liquid anticalcification treatment solution. However, other approaches could also be taken, such as vapor, plasma, and/or cryogenic application. Irrespective of the method of exposure, the time period should be sufficient to inhibit calcification, but not so long as to cause irreparable dehydration of the tissue by any of the constituents of the anticalcification treatment solution. In certain embodiments, the biomaterial is shaken or otherwise agitated during exposure to the anticalcification treatment solution in order to facilitate greater penetration of the constituents of the solution into the biomaterial. Shaking can be accomplished in any convenient manner, such as through use of an orbital shaker or shaker stand, or by manual agitation.

In some instances, it will be preferred to formulate an anticalcification treatment solution that is buffered in an aqueous solvent system, for example to a pH between about 6.0 and 8.0, preferably to a pH between about 7.0 and 7.6. Suitable buffers for use in this regard include buffers which have a buffering capacity sufficient to maintain a physiologically acceptable pH and do not cause any deleterious effects to the biomaterial or interfere with the treatment process being performed. Illustrative buffers include phosphate-buffered saline (PBS), organic buffers, such as N-N-2-hydroxyethylpiperzine-N′-2-ethanesulfonic acid (HEPES) and morpholine propanesulphonic acid (MOPS), and buffers which include borate, bicarbonate, carbonate, cacodylate, and the like. Many additional aqueous and other buffering systems suitable for use in the present invention will be apparent to the skilled artisan.

The biomaterial that has been treated with an anticalcification treatment solution may be rinsed prior to implantation or storage to remove any undesired and/or deleterious components produced or used in the biomaterial treatment protocol, such as cellular debris or aldehyde fragments from an aldehyde pretreatment. As used herein, the term “rinse” includes subjecting the biomaterial to a rinsing solution, including continuously or by batch processing, wherein the biomaterial is placed in a rinsing solution which may be periodically removed and replaced with fresh solution at predetermined intervals. During rinsing, the tissue is preferably shaken, or intermittently stirred, to ensure even distribution of the rinse solution. Illustratively, a rinse may comprise soaking the biomaterial in fresh rinsing solution which is replaced several times over a period of about an hour or less. Alternatively, the rinsing solution may be replaced at intervals of several hours or more over a longer rinse period, such as about 24 hours. Exemplary rinsing solutions include physiologically suitable solutions, such as water, saline, PBS, HEPES buffered saline, ringers lactate (pH 7.4), sodium bicarbonate (pH 7.4), tris (pH 7.4), imidazole (pH 7.4), and the like.

Subsequent to rinsing, the treated biomaterial is ready for implantation or may be sterilized and stored until use. Storage in standard glutaraldehyde solutions of the type typically used for long-term storage of clinical-grade bioprostheses may partially reverse the beneficial effects achieved by the treatment method of the present invention. Thus, it may be advantageous to store the treated biomaterial in an alcohol- or polyol-containing solution, such as an alcohol-glutaraldehyde solution, preferably under conditions which maintain calcification inhibition properties of the treated material.

In other embodiments of the invention, biomaterials which have been treated in accordance with the method of the invention are stored in an aldehyde-free environment. For example, treated tissue may be placed in sterile bags and subjected to sterilizing radiation, such as gamma-radiation. Of course, the treatment method of the present invention will be compatible with many other known sterilizing preservatives and/or techniques which are known by those of skill in the art.

In additional embodiments, the anticalcification treatment solution of the present invention may further comprise one or more additional anticalcification agents, including but not limited to, a soluble salt of a metallic cation, such as Al⁺³ or Fe⁺³, preferably in a concentration range of 0.001M to 0.1M. Water soluble aluminum salts, for example, which are suitable additional anticalcification agents for use in the practice of the present invention, include without limitation, aluminum chlorate, aluminum lactate, aluminum potassium sulfate, aluminum sodium sulfate, aluminum sulfate, aluminum nitrate, and aluminum chloride. Also, water-soluble ferric salts, such as ferric chloride, ferric nitrate, ferric bromide, ferric sodium edentate, ferric sulfate, and ferric formate, are also within the contemplation of the invention. Of course, any salt of aluminum, or iron, which is soluble in the solvent system of the treatment solution, may be used in the practice of the invention.

Although not wishing to be bound by this theory, the following may explain, at least in part, certain advantages realized by employing anticalcification treatment solutions in accordance with the present invention. In living tissue and cells, the typical extracellular calcium concentration is about 1 mM and the intracellular calcium concentration is about 0.1 μM. This large concentration gradient of calcium between the extracellular and intracellular regions is maintained by biochemical metabolic energy-dependent pumps across the plasma membranes of cells. Upon fixation, these biochemical forces are not active, and this results in a high concentration of calcium throughout the fixed tissue matrix. Plasma membranes and membrane bound organelles are rich in phospholipids, which provide phosphorous for calcium phosphate formation. In the in vivo environment, the high concentration of calcium in the fixed tissue coupled with a source of phosphorous from lipids may favor conditions for calcium phosphate crystallization. However, the constituents of the anticalcification treatment solutions used in accordance with this invention can be highly effective in penetrating the tissue matrix, interacting with, and possibly facilitating the removal of, phospholipids and other cellular debris from the cross-linked biomaterial, thereby interfering with the ability of such components to contribute to the crystallization process.

Certain embodiments of the present invention are directed to methods of treating a bioprosthesis, whereby the bioprosthesis can be less cytotoxic after being contacted with an aqueous composition than before being contacted with the aqueous composition. Restated contact with the aqueous composition is effective to reduce the cytotoxicity of the bioprosthesis. The present invention is also directed to bioprostheses suitable for implantation that are prepared using such methods. The method comprises providing a bioprosthesis that comprises an animal tissue that has been fixed with a chemical cross-linking agent, as described above, and before being contacted with the aqueous composition the bioprosthesis is cytotoxic upon implantation into an animal. Preferably the bioprosthesis is contacted with the aqueous composition at between about 30° C. and 60° C., and more preferably at about 37° C. Preferably it is contacted with the composition for between about 1 minute and 5 years, more preferably about 2 hours and 72 hours, and most preferably between about 48 and 68 hours. The temperature and time that the bioprosthesis is contacted with the aqueous composition is sufficient to reduce the cytotoxicity of the bioprosthesis upon implantation. In certain embodiments, the chemical cross-linking agent comprises an aldehyde, as described above. Certain fixed bioprostheses commercially available are also stored in buffered glutaraldehyde solution. However glutaraldehyde is cytotoxic and cannot typically be completely removed during implantation procedures. The bioprosthesis is typically immersed in the aqueous composition, and the bioprosthesis is removed from the composition before being implanted in a patient. Preferably the bioprosthesis is rinsed, as described above, before being implanted.

The aqueous composition comprises at least one salt and a C₁-C₃ alcohol. The C₁-C₃ alcohol can, for example, be selected from the group consisting of isopropyl alcohol, ethyl alcohol, methyl alcohol, and mixtures thereof. Preferably the C₁-C₃ alcohol is ethanol. The salt can be an inorganic or organic salt. Preferably the aqueous composition comprises an organic salt, such as triethanolamine hydrochloride or N-2-Hydroxyethylpiperazine-N′-2-ethanesulfonic acid hydrochloride. In certain embodiments the aqueous composition comprises the inorganic salt, sodium chloride. Preferably the aqueous composition comprises between about 0.1 and 1 wt % of the sodium chloride. More preferably the aqueous comprises between about 0.7 and 0.8 wt % sodium chloride, and most preferably about 0.8 wt %.

In certain embodiments, the cytotoxicity reducing aqueous composition further comprises a C₄-C₃₆ alcohol, more preferably a C₆-C₁₈ alcohol. The C₄-C₃₆ alcohols suitable for use in the present invention can be linear or branched, and can be mono-hydroxy alcohols or polyols. It is preferred that the C₆-C₁₈ alcohol is selected from the group consisting of heptanol, octanol, nonanol, 1,2-octanediol, 1,8-octanediol, 1,10-decanol, 1,10-dodecanol, 1,2-dihydroxydecane, 1,2-dihydroxydodecane, and mixtures thereof. More preferably the C₆-C₁₈ alcohol is 1,2-octanediol.

In a preferred embodiment, the aqueous composition comprises between about 0.1 and is 10% by volume of the C₆-C₁₈ alcohol, between about 15 and 25% by volume of the C₁-C₃ alcohol, and between about 65 and 85% by volume of an aqueous salt solution (e.g., the composition can comprise up to about 5% octanediol, 20.9% ethanol, and 74.1% HEPES). In a preferred embodiment the aqueous composition can comprise a trace amount of 1,2-octanediol (about 0.1% by volume), about 22% ethanol, and the remainder being HEPES buffer.

The salt solution in certain embodiments can consist essentially of sodium chloride and water, such that the concentration in the aqueous alcohol solution that comprises the salt solution is between about 0.1 and 1 wt %; more preferably between about 0.7 and 0.8 wt %; most preferably about 0.8 wt %. The salt solution can, in certain embodiments, comprise a chemical buffer, and the salt solution can be selected from the group consisting of triethanolamine buffer, HEPES buffered (N-2-Hydroxyethylpiperazine-N′-2-ethanesulfonic acid buffered) saline, Tris buffered (Tris-(hydroxymethyl)aminomethane buffered) saline, PBS (phosphate buffered saline), HEPES buffer, Pipes buffer (Piperazine-N,N′-bis(ethanesulfonic acid) buffer), Tris buffer, and Bis-Tris buffer (bis(2-hydroxyethyl)-imino-tris(hydroxymethyl)methane buffer). HEPES buffered saline can have a pH of between about 7.05 and 7.4. Tris buffered saline can have a pH of between about 7.4 and 7.5. HEPES buffer has a buffer range of between about pH 6.8 to 8.2, and a working concentration range of between about 20 mM and 200 mM. Tris buffer has a buffer range of between about pH 7.3 and 7.8, and a working concentration range of between about 20 mM and 200 mM. Phosphate buffered saline has a buffer range between about pH 7.2 and 7.4, and a working concentration range of between about 10 mM and 200 mM. Pipes buffer has a buffer range of between about pH 6.1 and 7.5, and a working concentration range of between 20 mM and 100 mM. Bis-Tris has a buffer range of about pH 5.8 and 7.3, and a working concentration range of between about 20 mM and 200 mM. Preferably the salt solution comprises an organic salt, such as triethanolamine hydrochloride or HEPES. Furthermore, preferably the concentration of the chemical buffer salt in the salt solution is between about 10 mM and 200 mM. Preferably, when the salt solution has a pH of between about 6.8 and 7.2, more preferably about 7, especially when the salt solution is a chemical buffer solution.

Thus, in a preferred embodiment, a bioprosthesis that comprises a fixed animal tissue that has been fixed with an aldehyde, is contacted with an aqueous composition comprising between about 0.1 and 10% by volume of a C₇-C₉ alcohol, between about 15 and 25% by volume of a C₁-C₃ alcohol, and between about 65 and 85% by volume of an aqueous salt solution is selected from the group consisting of sodium chloride solution, triethanolamine buffer, sodium chloride solution, HEPES buffered saline, Tris buffered saline, phosphate buffered saline, HEPES buffer, Pipes buffer, Tris buffer, and Bis-Tris buffer. Preferably the aqueous composition comprises up to about 5% by volume 1,2-octanediol, and 95% (22% ethanol and 73% HEPES buffer). The bioprosthesis is less cytotoxic after being contacted with the aqueous composition than before being contacted with the aqueous composition.

In certain embodiments the aqueous composition can comprise between about 20 to 25% by volume ethanol and between about 70 to 80% by volume aqueous buffer. More preferably, the aqueous composition comprises about 22% ethanol and about 78% PBS or HEPES. In other embodiments, the aqueous composition can comprise between about 45 to 55% by volume isopropanol and between about 45 to 55% by volume HEPES buffer. More preferably, the aqueous composition comprises about 50% isopropanol and about 50% HEPES buffer.

The bioprosthesis and the aqueous composition can, in certain embodiments, be packaged in a container; and the bioprosthesis, the aqueous composition, and the container can be exposed to sterilizing radiation. In certain embodiments, the bioprosthesis can be removed from contact with the aqueous composition and implanted in a human patient. Preferably the bioprosthesis treated using cytotoxicity-reducing compositions of the present invention retains favorable properties (e.g., mechanical durability and biostability, among others) that the bioprosthesis had prior to such treatment (e.g., contacted with the aqueous composition).

Certain embodiments of the present invention are directed to methods of treating a implantable medical device, whereby the implantable medical device can comprise less of a viral contaminant after being contacted with an aqueous composition than before being contacted with the aqueous composition. Other embodiments of the present invention are directed to implantable medical devices suitable for implantation that are prepared using such methods. In certain embodiments, the viral contaminant is capable of infecting an animal host, especially a human patient. Preferably the implantable medical device is contacted with the aqueous composition (e.g., viral contaminant reducing composition) at between about 30° C. and 60° C., and more preferably at about 37° C. Preferably it is contacted with the composition for between about 1 minute and 5 years, more preferably about 2 hours and 72 hours, and most preferably between about 48 and 68 hours. The temperature and time that the implantable medical device is contacted with the aqueous composition is sufficient to reduce the viral contamination of the implantable medical device. Preferably the amount of viral contaminant is reduced by at least about one log by contacting the implantable medical device with the aqueous composition. More preferably the amount of viral contaminant is reduced by between about one and five logs by contacting the implantable medical device with the aqueous composition. The amount of viral contaminant as used in the present invention refers to the number of virus particles in/on an implantable medical device that are capable of infecting a cell. The implantable medical device comprises at least one viral contaminant. Examples of viruses that can contaminate the device are murine leukemia virus, influenza A virus, porcine parvovirus, pseudorabies virus, HIV, hepatitis A-C and reovirus, among others. The viral contaminant can be any human virus, when the implantable medical device comprises human tissue. The foregoing viruses are exemplary, and not exhaustive. In certain embodiments the implantable medical device can be a bioprosthesis comprising an animal tissue as described above. The animal tissue can optionally be fixed, as described above. In other embodiments the implantable device does not comprise a tissue, such as a mechanical heart valve or a catheter, among others. In certain embodiments, the method can further comprise implanting the device in a patient.

The implantable medical device is contacted with an aqueous composition comprising at least one salt, a C₄-C₃₆ alcohol, and a C₁-C₃ alcohol, whereby the implantable medical device comprises less viral contaminant after being contacted with the aqueous composition than before being contacted with the aqueous composition. Preferably the C₄-C₃₆ alcohol, the C₁-C₃ alcohol, and the salt are as described above. Preferably the C₄-C₃₆ alcohol is a C₆-C₁₈ alcohol that is selected from the group consisting of heptanol, octanol, nonanol, 1,2-octanediol, 1,8-octanediol, 1,10-decanol, 1,10-dodecanol, 1,2-dihydroxydecane, 1,2-dihydroxydodecane, and mixtures thereof. More preferably the C₄-C₃₆ alcohol is 1,2-octanediol. The C₄-C₃₆ alcohols can be linear or branched, and can be mono-hydroxy alcohols or polyols.

In a preferred embodiment the aqueous composition comprises between about 0.1 and 10% by volume of the C₆-C₁₈ alcohol, between about 15 and 45% by volume of the C₁-C₃ alcohol, and between about 45 and 85% by volume of an aqueous salt solution, and more preferably up to about 5% by volume of the C₆-C₁₈ alcohol, between about 20 and 45% by volume of the C₁-C₃ alcohol, and between about 50 and 75% by volume of an aqueous salt is solution. Preferably the C₄-C₃₆ alcohol, the C₁-C₃ alcohol, and the aqueous alcohol solution are as described above. In certain embodiments the aqueous salt solution comprises a chemical buffer, and can be selected from triethanolamine buffer, HEPES buffered saline, Tris buffered saline, phosphate buffered saline, HEPES buffer, PIPES buffer, Tris buffer, and Bis-Tris buffer, among others. More preferably the aqueous salt solution is HEPES buffer. The C₁-C₃ alcohol as described above. In certain embodiments the implantable medical device is treated with at least one antiviral treatment before being contacted with the aqueous composition. Preferably the implantable medical device comprises an animal tissue. The at least one antiviral treatment can comprise contacting the implantable medical device with an aldehyde. Alternatively the additional antiviral treatment can comprise treatments known in the art such as chemical or solvent/surfactant based treatments. Thus, a chemical treatment of an implantable medical device can be followed by a solvent/surfactant treatment, which is then followed with an aqueous composition of the present invention.

The implantable medical device and the aqueous composition can, in certain embodiments, be packaged in a container; and the implantable medical device, the aqueous composition, and the container can be exposed to sterilizing radiation. In certain embodiments the implantable medical device can be removed from contact with the aqueous composition and implanted in a human patient. Preferably the implantable medical device treated using viral contamination-reducing compositions of the present invention retains favorable properties (e.g., mechanical durability and biostability, among others) that the implantable medical device had prior to such treatment (e.g., contacted with the aqueous viral-reducing composition).

In a particular, preferred embodiment, an implantable medical device that comprises at least one viral contaminant is contacted with an aqueous composition comprising about 0.1 and 10% by volume of a C₇-C₉ alcohol, between about 15 and 45% by volume of a C₁-C₃ alcohol, and between about 45 and 85% by volume of an aqueous salt solution selected from the group consisting of sodium chloride solution, triethanolamine buffer, sodium chloride solution, HEPES buffered saline, Tris buffered saline, phosphate buffered saline, HEPES buffer, Pipes buffer, Tris buffer, and Bis-Tris buffer. Preferably the aqueous composition comprising up to about 5% octanediol, 40% ethanol, and 55% HEPES. The implantable medical device comprises at least about one log less viral contaminant after being contacted with the aqueous composition than before being contacted with the aqueous composition. More preferably the implantable medical device comprises between about one and five logs less viral contaminant after being contacted with the aqueous composition than before being contacted with the aqueous composition.

In a particularly preferred embodiment, a sterile heart valve bioprosthesis available commercially, is transferred from its original shipping solution (0.2% buffered glutaraldehyde) to a new container containing an aqueous composition comprising about 5% 1,2-octanediol, 40% ethanol and 55% HEPES buffer. The container is closed and transferred to an incubator at about 37° C. for up to about 68 hours. The valve is shaken during treatment in the incubator. Shaking during the process can assist in lipid extraction from the bioprosthesis/penetration of treatment solution into the bioprostheis. After the treatment, the aqueous composition is removed, and replaced with alcohol solution comprising about 22% alcohol (e.g., ethanol solution) under aseptic conditions. The container is closed. The bioprosthesis stored in the alcohol solution can be implanted and it can be shipped in the alcohol solution. The treated valve can be durable, non-calcific, non-cytotoxic and have a reduced viral load. The following examples are provided to demonstrate certain illustrative embodiments of this invention. It should be appreciated by is those skilled in the art that the techniques disclosed in the examples which follow represent those found by the inventors to function in the practice of the invention and thus can be considered to constitute examples of illustrative modes for its practice. However, those skilled in the art should, in light of the present disclosure, appreciate that many changes can be made in the specific embodiments which are disclosed and still obtain a like or similar result without departing from the spirit and scope of the invention.

### 4.0 EXAMPLES

### 4.1 Example 1

### Treatment of Aldehyde-Fixed Tissue with Higher Alcohols

Bovine pericardium was obtained fresh from the abattoir, trimmed to remove excess fat, and stored in a high osmolarity solution until use. Prior to fixation, the tissue was rinsed thoroughly in phosphate buffered saline (PBS) having a pH of 7.3-7.4. A 0.25% solution of glutaraldehyde was prepared by adding 2.5 ml of a 50% glutaraldehyde solution (Aldrich Chemical) to 500 ml using PBS. Fifteen 1 cm×1 cm samples of bovine pericardium tissue were added to the glutaraldehyde solution and the tube was stored at room temperature for 7 days.

In a class 100 laminar flow bench, glutaraldehyde fixed bovine pericardium pieces were washed with sterile PBS (3 washes, 10 minutes each). The samples were then immersed in a sterile filtered solution of 40% ethanol, 5% octanol, and 55% water and treated for 24 hours at room temperature. The tissue was then washed with sterile PBS (3 washes, 10 minutes each), and in sterile filtered 45% ethanol in PBS for about 30 minutes. The samples were stored in 40 ml PBS for about 1 day prior to using them for rat implantation studies.

In a separate experiment, five 1cm×1cm samples of glutaraldehyde fixed bovine pericardium tissue (0.25% glutaraldehyde, 16 hrs) were treated with a solution comprised of 40% ethanol, 5% octanol and 55% water for 30 minutes. An additional 5 samples were treated in the solution for 24 hrs. After treatment, the samples were washed with PBS (30ml×3) and stored in 45% ethanol. The samples were then analyzed to evaluate the presence of extractable proteins and to determine shrinkage temperatures.

### 4.1.1 Evaluation of Extractable Proteins and Shrinkage Temperatures

Cross-linking biological tissue results in less extractable protein within the material. Protein extraction assays were performed by extracting 10-20 mg of tissue with 10-20 μl of an extraction solution containing 50 mM Tris-HCl, 10% glycerol, 4% mercaptoethanol, 1% sodium dodecyl sulfate, 0.5M NaCl and 0.01% bromophenol blue. The extracted solution was then analyzed on a 4-20% acrylamide:bisacrylamide (37.5:1) Mini-PROTEAN II ready Gel (Biorad Inc).

The shrinkage temperatures of the treated tissues were also determined using standard differential scanning calorimetric analysis. Typically, 2-10 mg of tissue was heated at the rate of 10° C. per minute under nitrogen atmosphere. The onset of the endotherm observed at about 60-90° C. is conventionally attributed to a shrinkage transition, and was used as the shrinkage temperature. An increase in the shrinkage temperature is an indication that cross-linking has occurred.

The results of the extractable protein and shrinkage temperature determinations are summarized in Table 1 below:

TABLE 1

| Glutaraldehyde | Alcohol | Extractable | Shrink |
|---|---|---|---|
| Treatment | Treatment | Proteins | Temp. (° C.) |
| None | None | Yes | 66.3 |
| 0.25%, 24 hrs. | None | No | 79.2 |
| 0.25%, , 24 hrs. | 40% EtOH | No | 79.9 |
| | 5% Octanol, 1 hr. | | |
| 0.25%, , 24 hrs. | 40% EtOH | No | 80.2 |
| | 5% Octanol, 24 hr. | | |

From these results, it is clear that the treatment caused no degradation of the glutaraldehyde fixed tissue, as evidenced by the absence of extractable proteins. Moreover, neither the 1 hour nor the 24 hour treatments substantially effected shrink temperature values, indicating that the treatment did not alter physical properties of the glutaraldehyde fixed tissue.

### 4.1.2 Evaluation of Calcification Following In Vivo Implantation

Prior to implantation, the samples were rinsed 3 times for 3 minutes each in 500 ml containers of sterile PBS, accompanied by gentle agitation. Treated and untreated specimens were implanted subcutaneously using standard surgical procedures approximately 1 cm from the abdominal midline in 3 week old Sprague-Dawley rats. The implanted tissue was retrieved after 60 days.

Upon their removal, the tissue samples were processed using standard histological methods and stained with H&E, Van Kosa and Masson's trichrome. Van Kosa stain identifies calcification of the tissue. The extent of calcification by Van Kosa stain was graded from 0 (none) to 5 (severe).

The calcium content of the retrieved samples was determined by hydrolyzing the samples under acidic conditions and analyzing the digested samples using standard inductively coupled plasma (ICP) emission spectrophotometry. Typically, about 0.5 g of the explanted tissue was dried, weighed and hydrolyzed under acidic conditions. The resulting digested sample was diluted with water and analyzed using an ICP spectrophotometer (Varian Inc.; Liberty 100/200 ICP-OCS).

The results of these experiments are summarized in Table 2 below:

TABLE 2

| Glutaraldehyde | Alcohol | Calcium | Average |
|---|---|---|---|
| Treatment | Treatment | (μg/mg dry tissue) | Van Kosa Grading |
| 0.25%, 14 days | None | 201 | 5 |
| 0.25%, 14 days | 45% EtOH | 168 | 5 |
| | 24 hrs | | |
| 0.25%, 7 days | 40% EtOH | 0.72 | 0 |
| | 5% Octanol | | |
| | 24 hrs | | |

The tendency of glutaraldehyde fixed tissue to calcify in the rat model is well documented in the literature, and this was confirmed by our experiments. However, the glutaraldehyde fixed samples treated with an anticalcification treatment solution containing a higher alcohol (e.g., octanol) exhibited a significant reduction in calcification compared to those not treated. Samples treated with a 45% ethanol solution for 24 hours at room temperature showed values similar to the control samples.

### 4.2 Example 2

### Treatment of Aldehyde-Fixed Tissue with 1,2-Octanediol and N-Methyl Pyrolidinone

In a class 100 laminar flow bench, pieces of glutaraldehyde-fixed bovine pericardium tissue (Mitroflow Inc.; Richmond, British Columbia, Canada) porcine cusp tissue (Labcor; Belo Horizonte, Brazil) and porcine wall tissue (Labcor Inc.) were transferred into sterile tubes containing 1,2-octanediol solutions (5% 1,2-octanediol (Aldrich Chemical), 40% ethanol and 55% 10 mM HEPES buffer). The tubes were transferred to a 37° C. incubator and maintained at 37° C. with gentle agitation for about 16 hours. After the treatment, the samples were transferred to solutions comprising 22% ethanol in 10 mM HEPES and stored for 14 days at 4° C. The final tissue to volume ratio for all treatments was approximately 27 ml/g.

For N-methyl pyrolidinone (NMP) treatments, pieces of glutaraldehyde-fixed bovine pericardium tissue (Mitroflow Inc.), porcine cusp tissue (Labcor Inc.) and porcine wall tissue (Labcor Inc.) were transferred into sterile tubes containing NMP. The tubes were incubated at room temperature for about 16 hours with occasional manual agitation. After the treatment, the tissue samples were transferred to 22% HEPES-buffered ethanol solutions and stored for 14 days at 4° C.

### 4.2.1 Evaluation of Calcification Following In Vivo Implantation

Samples treated with the 1,2-octanediol solutions and with NMP, as well as untreated samples of each tissue type, were provided to Charles Rivers Laboratories (Wilmington, Mass.) for implantation into rats. Seven rats per treatment group were analyzed. Prior to implantation, the tissue samples were rinsed for 3 minutes ×3 in sterile PBS, accompanied by gentle agitation. The samples were implanted subcutaneously approximately 1 cm from the abdominal midline in 3 week old Sprague-Dawley rats and retrieved after 60 days of implantation. Unimplanted samples (one per tissue type per treatment) were used as unimplanted controls. After retrieval, the samples were analyzed for their calcium and phosphorus contents using a standard ICP methodology.

The results of these experiments are summarized below in Table 3.

TABLE 3

| Post-Fixation | | Calcuim | Phosphorus |
|---|---|---|---|
| Treatment | Tissue Type | (μg/mg tissue) | (μg/mg tissue) |
| None | Bovine | 259.6 | 130.4 |
| | Pericardium | | |
| None | Porcine Cusp | 348.0 | 174.8 |
| None | Porcine Wall | 199.2 | 102.2 |
| NMP | Bovine | 8.0 | 3.4 |
| | Pericardium | | |
| NMP | Porcine Cusp | 14.7 | 7.9 |
| NMP | Porcine Wall | 111.9 | 55.2 |
| 1,2-octanediol | Bovine | 3.4 | 0 |
| | Pericardium | | |
| 1,2-octanediol | Porcine Cusp | 44.1 | 21.9 |
| 1,2-octanediol | Porcine Wall | 106.4 | 53.0 |

Unimplanted controls had very low calcium and phosphorus levels (not shown). From the above table, however, it can be seen explanted tissue samples that had not had not been treated with an anticalcification treatment solution had very high levels of calcium and phosphorus. This was observed irrespective of the tissue type. On the other hand, explanted tissues that had been treated with either a 1,2-octanediol solution or with NMP had significantly reduced calcium and phosphorus levels. Interestingly, although the levels were reduced for all tissue types, the effect was most pronounced with bovine pericardium.

Explanted tissue samples were also sectioned, stained with H&E, and evaluated histologically for inflammation, vascularization and collagen organization. The 1,2-octanediol-treated samples, the NMP-treated samples, and the control samples had similar histological grading, indicating that the anticalcification treatments did not alter the biological response by the host animal.

### 4.2.2 Analysis of Extractable Proteins and Shrinkage Temperatures

For these experiments, bovine pericardium samples were placed in 0.25% solutions of glutaraldehyde in PBS where they remained at room temperature for about 7 days. The cross-linked tissues were then subjected to either 1,2-octanediol or NMP treatments, as described above. The treated samples were then analyzed for extractable proteins and to determine shrinkage temperatures.

In addition, enzymatic digestion assays were performed as follows. Tissue samples were digested after thermal denaturation for 10 minutes at 80° C. in 4 mg/ml pepsin (Sigma Chemical, St. Louis, Mo.) in 10 mM HCl for 4 hours at 37° C. Enzyme: tissue ratios (weight: wet weight) were 1:2500). Following centrifugation at 4° C. for 5 minutes at 13,000 rpm (30,000 ×g), reaction supernatants were used for gel electrophoresis.

The results of these experiments are summarized below in Table 4.

TABLE 4

| | | Extractable | Extractable | |
|---|---|---|---|---|
| | | Protein in | Protein | Shrink |
| Glutaraldehyde | Post-Fixation | Extraction | after Pepsin | Temperature |
| Treatment | Treatment | Assay | Digestion | (° C.) |
| No | None | Yes | Yes | 61.8 |
| Yes | None | No | No | 87.0 |
| Yes | NMP | No | No | 85.6 |
| Yes | 1,2- | No | NO | 86.0 |
| | octanediol | | | |

These results demonstrate that for both anticalcification treatments (1,2-octanediol and NMP), there was no significant effect on shrinkage temperature when compared with untreated tissue, suggesting no significant change in the cross-linking status of the tissue had occurred as a result of the anticalcification treatments. Furthermore, both the treated and untreated samples failed to show any extractable proteins following the protein extraction and pepsin digestion assays, indicating that the anticalcification treatments did not adversely affect the biostability of the tissue.

### 4.3 Example 3

### Treatment of Aldehyde-Fixed Tissue with Lower Alcohol Solutions

In a class 100 laminar flow bench, pieces of glutaraldehyde-fixed bovine pericardium tissue (Mitroflow Inc.) were transferred into sterile tubes containing a 45% solution of HEPES-buffered ethanol (45% ethanol, 55% 10 mM HEPES buffer). The tubes were transferred to a 37° C. incubator and maintained at 37° C. with gentle agitation for about 16 hours. After the treatment, the samples were transferred to fresh solution of 45% HEPES-buffered ethanol and stored for 14 days at room temperature (˜25° C.). The final tissue to volume ratio for all treatments was approximately 27 ml/g.

### 4.3.1 Evaluation of Calcification following In Vivo Implantation

Samples treated with the 45% ethanol solution, as well as untreated samples of each tissue type, were provided to Charles Rivers Laboratories (Wilmington, Mass.) for implantation into rats. Seven rats per treatment group were analyzed. Prior to implantation, the tissue samples were rinsed for 3 minutes ×3 in sterile PBS, accompanied by gentle agitation. The samples were implanted subcutaneously approximately 1 cm from the abdominal midline in 3 week old Sprague-Dawley rats and retrieved after 60 days of implantation. Unimplanted samples (one per tissue type per treatment) were used as unimplanted controls. After retrieval, the samples were analyzed for their calcium and phosphorus contents by ICP.

The results of these experiments are summarized below in Table 5.

TABLE 5

| Post-Fixation | | Calcuim | Phosphorus |
|---|---|---|---|
| Treatment | Tissue Type | (μg/mg tissue) | (μg/mg tissue) |
| None | Bovine | 259.6 | 130.4 |
| | Pericardium | | |
| 45% Ethanol | Bovine | 3.4 | 0.0 |
| | Pericardium | | |

This data demonstrates that the implanted tissue samples that did not receive any anticalcification treatment showed high levels of calcium and phosphorus. However, tissue samples treated with the ethanol solution showed a significant reduction in both levels. After the 60 day implantation period, unimplanted control samples had very low levels of calcium and phosphorus (not shown).

Thus, lower alcohol solutions having below 50% by volume of alcohol can reduce calcification under appropriate treatment conditions, for example by using elevated temperature to improve the efficacy of the treatment. Other solutions containing less than 50% by volume of a lower alcohol, for example methanol or isopropanol, could also be used.

### 4.4 Example 4

### Evaluation of Cytoxicity Reducing Treatments of Aldehyde-Fixed Tissues

Fixed porcine valves that had been removed from a 0.2% glutaraldehyde storage solution were subjected to treatment, soaks, and/or rinses as specified in Table 6, below, and then extracted and tested for cytotoxicity using an elution methodology.

Some of the valves were rinsed three times (3×) for a minimum of two minutes in physiological saline, or were not rinsed/soaked at all. The major difference between a rinse and a soak is that a “rinse” is defined as constant agitation during the time duration. A “soak” is defined as no agitation during the time duration.

After the rinses or soaks, the porcine valves were extracted in MEM (mammalian cell culture medium) or SC (sodium chloride) at 37° C. for 24 hours at an extraction ratio of 4 g:20 ml (USP <87>extraction ratio).

The MEM extracts were used to directly dose L-929 mouse fibroblast cells, as indicated by USP <87>. The SC extracts of the devices were diluted 1:4 (1 part SC test extract and 3 parts MEM), as indicated by USP <87>, and the diluted extracts were used to directly dose L-929 mouse fibroblast cells. The samples were tested in duplicate per USP or in triplicate per ISO 10993-5.

Test controls were similarly prepared. The extracts were placed on L-929 mouse fibroblast cells and incubated 48 hours at 37° C. After incubation, the cell cultures were examined microscopically at 100× magnification to determine cell morphology. Observations were evaluated using USP 24 guidelines.

TABLE 6
OVERVIEW OF TREATMENTS AND RINSES FOR PORCINE VALVES

| Sample | | | |
|---|---|---|---|
| number | Treatment(s) | Rinse(s) | Extraction Method |
| 1 | No treatment | No Rinse | 37° C., 24 hr in MEM |
| 2 | No treatment | No Rinse | 37° C., 24 hr in SC |
| 3 | No treatment | 3X rinse in physiological saline, 2 | 37° C., 24 hr in MEM |
| | | minutes each | |
| 4 | No treatment | 3X rinse in physiological saline, 2 | 37° C., 24 hr in SC |
| | | minutes each | |
| 5 | Treated with 5% octanediol, 95% | No Rinse | 37° C., 24 hr in MEM |
| | (HEPES buffered 22% ethanol) | | |
| 6 | Treated with 5% octanediol, 95% | No Rinse | 37° C., 24 hr in SC |
| | (HEPES buffered 22% ethanol) | | |
| 7 | Treated with 5% octanediol, 95% | One 2 minute soak in | 37° C., 24 hr in MEM |
| | (HEPES buffered 22% ethanol) | physiological saline | |
| 8 | Treated with 5% octanediol, 95% | One 2 minute soak in | 37° C., 24 hr in SC |
| | (HEPES buffered 22% ethanol) | physiological saline | |
| 9a | Treated with 5% octanediol, 95% | 3X rinse in physiological saline, 2 | 37° C., 24 hr in SC |
| | (HEPES buffered 22% ethanol) | minutes each | |
| 9b | Treated with 5% octanediol, 95% | 3X rinse in physiological saline, 2 | 37° C., 24 hr in MEM |
| | (HEPES buffered 22% ethanol) | minutes each | |
| 10 | Treated with HEPES buffered | No Rinse | 37° C., 24 hr in MEM |
| | 22% ethanol | | |
| 11 | Treated with HEPES buffered | One 2 minute soak in | 37° C., 24 hr in MEM |
| | 22% ethanol | physiological saline | |

### 4.4.1 Evaluation of Cytotoxicity After Contact with Treatment Composition

Table 7 provides a summary of the USP cytotoxicity results. The sample meets the USP and ISO requirements of the cytotoxicity test if the response to the sample preparation is not greater than a grade 2 (e.g., mildly reactive).

TABLE 7
CYTOTOXICITY RESULTS

| Sample | | | Extraction | |
|---|---|---|---|---|
| number | Treatment | Rinse(s) | Vehicle | USP Cytotoxicity Results |
| 1 | No treatment | No rinse | MEM | Toxicity greater than grade 2 observed. |
| | | | | Does not meet USP and ISO |
| | | | | requirements. |
| 2 | No treatment | No rinse | SC | Toxicity greater than grade 2 observed. |
| | | | | Does not meet USP and ISO |
| | | | | requirements. |
| 3 | No treatment | 3X rinse in | MEM | Toxicity greater than grade 2 observed. |
| | | physiological saline, 2 | | Does not meet USP and ISO |
| | | minutes each | | requirements. |
| 4 | No treatment | 3X rinse in | SC | Toxicity greater than grade 2 observed. |
| | | physiological saline, 2 | | Does not meet USP and ISO |
| | | minutes each | | requirements. |
| 5 | Treated with 5% | No rinse | MEM | Toxicity greater than grade 2 observed. |
| | octanediol, 95% | | | Does not meet USP and ISO |
| | (HEPES buffered | | | requirements. |
| | 22% ethanol) | | | |
| 6 | Treated with 5% | No rinse | SC | No cell lysis or toxicity greater than 2 |
| | octanediol, 95% | | | was observed. Does meet USP and ISO |
| | (HEPES buffered | | | requirements. |
| | 22% ethanol) | | | |
| 7 | Treated with 5% | One 2 minute soak in | MEM | Toxicity greater than grade 2 observed. |
| | octanediol, 95% | physiological saline | | Does not meet USP and ISO |
| | (HEPES buffered | | | requirements. |
| | 22% ethanol) | | | |
| 8 | Treated with 5% | One 2 minute soak in | SC | No cell lysis or toxicity greater than 2 |
| | octanediol, 95% | physiological saline | | was observed. Does meet USP and ISO |
| | (HEPES buffered | | | requirements. |
| | 22% ethanol) | | | |
| 9a | Treated with 5% | 3X rinse in | SC | No cell lysis or toxicity greater than 2 |
| | octanediol, 95% | physiological saline, 2 | | was observed. Does meet USP and ISO |
| | (HEPES buffered | minutes each | | requirements. |
| | 22% ethanol) | | | |
| 9b | Treated with 5% | 3X rinse in | MEM | Toxicity greater than grade 2 observed. |
| | octanediol, 95% | physiological saline, 2 | | Does not meet USP and ISO |
| | (HEPES buffered | minutes each | | requirements. |
| | 22% ethanol) | | | |
| 10 | Treated with | No rinse | MEM | No cell lysis or toxicity greater than 2 |
| | HEPES buffered | | | was observed. Does meet USP and ISO |
| | 22% ethanol | | | requirements. |
| 11 | Treated with | One 2 minute soak in | MEM | No cell lysis or toxicity greater than 2 |
| | HEPES buffered | physiological saline | | was observed. Does meet USP and ISO |
| | 22% ethanol | | | requirements. |

Untreated devices without a rinse prior to testing were cytotoxic (samples 1 and 2). Untreated devices rinsed three times for two minutes prior to testing were cytotoxic (samples 3 and 4). Devices treated with 5% 1,2-octanediol and 95% (HEPES buffered 22% ethanol) without a rinse prior to testing were cytotoxic when extracted in MEM and were not cytotoxic when extracted in SC (samples 5 and 6). Devices treated with 5% octanediol and 95% (HEPES buffered 22% ethanol) with or without a rinse prior to testing extracted with SC were nontoxic (samples 6, 8, and 9a). A two minute soak in physiological saline of devices that had been treated with 5% octanediol and 95% (HEPES buffered 22% ethanol) had no effect on cytotoxicity results based on MEM extracts (samples 5, 7, and 9b). Both MEM extracts from devices that were treated with compositions comprising ethanol and HEPES buffer and no octanediol were nontoxic (samples 10 and 1 1).

### 4.5 Example 5

### Evaluation of Release of Compounds After Contact with Treatment Compositions

An experiment was performed to asses the release of residual glutaraldehyde, a composition comprising 5% octanediol, 40% ethanol, and 55% HEPES, and ethanol in glutaraldehyde fixed porcine bioprostheses. For residuals analysis, three 27A Oxford™ bioprostheses (e.g., fixed porcine heart valve bioprostheses) that had been treated with a composition comprising 5% octanediol, 40% ethanol, and HEPES were rinsed a total of two minutes (without agitation). The amount of glutaraldehyde, a composition comprising 5% octanediol , 40% ethanol, and 55% HEPES, and ethanol were measured from this initial rinse.

After rinsing, the devices were subsequently rinsed in saline and tested for glutaraldehyde, the composition comprising 5% octanediol, 40% ethanol, and 55% HEPES, and ethanol at 2, 24, and 48 hours. The average residuals released from the devices after 48 hours were less than 0.47 mg for glutaraldehyde, 22.4 mg for the composition comprising 5% octanediol, 40% ethanol, and 55% HEPES and 588.9 mg for ethanol. Residuals test data are provided in Table 8.

TABLE 8
AVERAGE GLUTARALDEHYDE, ETHANOL AND TREATMENT
COMPOSITION RELEASED OVER TIME

| | Average | Average | Average Treatment Composition |
|---|---|---|---|
| Extraction | Glutaraldehyde | Ethanol | (5% octanediol + 40% |
| Time (hr) | (mg) | (mg) | ethanol + 55% HEPES) |
| 4 | <0.42 | 541.4 | 17.2 |
| 24 | <0.445 | 584.5 | 21.4 |
| 48 | <0.47 | 588.9 | 22.4 |
| Rinse | <0.15 | 725 | 13.8 |
| solution | | | |

Prior residual studies for Oxford™ bioprostheses that were not treated with the treatment composition (5% octanediol, 40% ethanol, and 55% HEPES) indicated that 9 mg of glutaraldehyde were released after 48 hours. When compared to the data in the table above, the amount of glutaraldehyde residuals released from the valves contacted with the treatment compositions after 48 hours was approximately 19 times less than the amount released from Oxford™ bioprostheses that had not been treated with the composition.

### 4.6 Example 6

### Evaluation of Viral Inactivation After Contact with Treatment Compositions

The purpose of this evaluation was to characterize viral inactivation of five viruses from two different processing steps (glutaraldehyde fixation, and treatment with storage in (5% octanediol, 40% ethanol, and 55% HEPES) used in the manufacture of the porcine aortic root bioprostheses and pericardium. Viruses that were used in the evaluation are listed in Table 9.

Representative samples of the glutaraldehyde fixed porcine heart valve and pericardium bioprostheses were spiked with each of the five viruses described below (Table 9) and the bioprostheses were processed through two separate manufacturing steps using process parameters detailed below, and then assayed at specific times for remaining virus titers (Table 10). Toxicity tests of solutions to indicator cells to determine proper dilution of inoculated product were performed. Representative samples of the porcine valve and pericardium material were inoculated with the specified virus and incubated at appropriate temperature and times, and plated on indicator cells.

The two processes that were evaluated during the inactivation study included (1) a 0.2% glutaraldehyde stage 2 tissue fixing process (60-days duration), and (2) a treatment process involving 5% octanediol, 40% ethanol, and 55% HEPES composition (68±4 hours duration at 37° C.␣2° C.). The minimum time and temperature tolerances for the 0.2% glutaraldehyde treatment step and the 5% octanediol, 40% ethanol, and 55% HEPES composition treatment were used.

TABLE 9
VIRUS CHARACTERISTICS

| | | | | Approx- | |
|---|---|---|---|---|---|
| | | Envel- | Ge- | imate | |
| Virus | Virus Family | oped | nome | size (nm) | Shape |
| Amphotropic | Retroviridae | yes | RNA | 80-130 | spherical |
| Murine | | | | | |
| Leukemia | | | | | |
| Virus | | | | | |
| (A-MuLV) | | | | | |
| Influenza A | Orthomyx- | yes | RNA | 80-120 | spherical |
| Virus | oviridae | | | | |
| (Inf A) | | | | | |
| Porcine | Parvoviridae | no | DNA | 18-26 | icosahedral |
| Parvovirus | | | | | |
| (PPV) | | | | | |
| Pseudorabies | Herpesviridae | yes | DNA | 150-200 | spherical |
| Virus (PrV) | | | | | |
| Reovirus | Reoviridae | no | RNA | 60-80 | icosahedral |
| Type 3 | | | | | |
| (REO-3) | | | | | |

Porcine aortic heart valves fixed with glutaraldehyde were treated with a solution comprising 5% by volume 1,2-octanediol, 40% ethanol, and 55% HEPES buffer (10 mM, pH 7.2) for 68 hours at 37° C.

The 0.2% glutaraldehyde process completely inactivated all viruses at 2 consecutive time 10 points. As a result the 60 day 0.2 glutaraldehyde incubation process was terminated at 47 days. At that time sensitivity of the assay was increased 10-fold from a 6 well indicator to a 60 well indicator. No viruses were present on the porcine valves or pericardium materials after 47 days of exposure to 0.2% glutaraldehyde. No viruses were detected on the glutaraldehyde fixed devices after 64 hours exposure to the 5% octanediol, 40% ethanol, and 55% HEPES composition.

TABLE 10
VIRAL LOG REDUCTION

| | 0.2% | Treatment with 5% | Cumulative |
|---|---|---|---|
| | Glutaraldehyde | octanediol + 95% | Log |
| | Treatment at 47 | (HEPES buffered | Reduction |
| Virus | Days¹ | 22% ethanol)) | Factor |
| Amphotropic | 4.15 | 4.42 | 8.57 |
| Murine Leukemia | | | |
| Virus (A-MuLV) | | | |
| Influenza A Virus | 3.5 | 3.69 | 7.19 |
| (Inf A) | | | |
| Porcine Parvovirus | 5.03 | 3.99 | 9.02 |
| (PPV) | | | |
| Pseudorabies Virus | 5.51 | 4.04 | 9.55 |
| (PrV) | | | |
| Reovirus Type 3 | 5.00 | 5.27 | 10.27 |
| (REO-3) | | | |

¹The average log reduction factor for porcine valve and pericardium material are reported.

It is known in the art that, when evaluating results of viral inactivation studies, clearance factors from sequential orthogonal processes may be combined to give a cumulative clearance factor or cumulative log reduction factor. Orthogonal processes are those, which clear virus by independent modes of action such as solvent/detergent and heat. The 0.2% glutaraldehyde agent and the elevated temperature and ethanol in the 5% octanediol, 40% ethanol, and 55% HEPES composition are independent modes of action, and therefore the clearance factors from these two processes can be combined to provide a cumulative log reduction for the porcine valves and pericardium.

The cumulative log reduction factor of the 0.2% glutaraldehyde and 5% octanediol, 40% ethanol, and 55% HEPES treatments demonstrate the ability of the two manufacturing processes to completely inactivate a selected panel of viruses. This demonstrates that possible adventitious agents, including viral contaminants that may be introduced by the starting material (e.g., tissue) or by manufacturing processes are not present in the device that is finally implanted.

The particular embodiments disclosed above are illustrative only, as the invention may be modified and practiced in different but equivalent manners apparent to those skilled in the art having the benefit of the teachings herein. More specifically, it will be apparent that certain agents which are chemically and/or physiologically related may be substituted for the agents described herein while the same or similar results would be achieved. Furthermore, no limitations are intended to the details of construction or design herein shown, other than as described in the claims below. It is therefore evident that the particular embodiments disclosed above may be altered or modified and all such variations are considered within the scope and spirit of the invention. Accordingly, the protection sought herein is as set forth in the claims below.

### 5.0 REFERENCES

The following references, to the extent that they provide exemplary procedural or other details supplementary to those set forth herein, are specifically incorporated herein by reference.
U.S. Pat. No. 5,746,775
U.S. Pat. No. 6,479,079
Girardot et al., J Biomed Mater Res (1995) 29: 793-801
Golomb et al., Am J Pathol (1987) 127: 122-130
Gott, J. P. et al.; Ann. Thorac. Surg. (1992) 53, 207-215
Levy et al., In: Williams D F, ed. CRC Critical Rev. in Biocompatibility, Vol. 2 (1986): 147-187
Thubrikar et al., J Thorac Cardiovasc Surg (1983) 86: 115-125
Zilla et al., J Heart Valve Dis (1997) 6: 492-501

What is claimed:

1. A method for treating a bioprosthesis, comprising:
providing a bioprosthesis that comprises an animal tissue that has been fixed with a chemical cross-linking agent, wherein the bioprosthesis is cytotoxic upon implantation into an animal, and
contacting the bioprosthesis with an aqueous composition comprising at least one salt and a C₁-C₃ alcohol, whereby the bioprosthesis is less cytotoxic after being contacted with the aqueous composition than before being contacted with the aqueous composition.

2. The method of claim 1, wherein the aqueous composition further comprises a C₄-C₃₆ alcohol.

3. The method of claim 2, wherein the C₄-C₃₆ alcohol is a C₆-C18 alcohol.

4. The method of claim 3, wherein the C₆-C₁₈ alcohol is selected from the group consisting is of heptanol, octanol, nonanol, 1,2-octanediol, 1,8-octanediol, 1,1 0-decanol, 1,10-dodecanol, 1,2-dihydroxydecane, 1,2-dihydroxydodecane, and mixtures thereof.

5. The method of claim 4, wherein the C₆-C₁₈ alcohol is 1,2-octanediol.

6. The method of claim 3, wherein the aqueous composition comprises between about 0.1 and 10% by volume of the C₆-C₁₈ alcohol, between about 15 and 25% by volume of the C₁-C₃ alcohol, and between about 65 and 85% by volume of an aqueous salt solution.

7. The method of claim 6, wherein the aqueous salt solution comprises a chemical buffer.

8. The method of claim 7, wherein the aqueous salt solution is selected from the group consisting of triethanolamine buffer, HEPES buffered saline, Tris buffered saline, phosphate buffered saline, HEPES buffer, PIPES buffer, Tris buffer, and Bis-Tris buffer.

9. The method of claim 8, wherein the aqueous salt solution is HEPES buffer.

10. The method of claim 1, wherein the C₁-C₃ alcohol is selected from the group consisting of isopropyl alcohol, ethyl alcohol, methyl alcohol, and mixtures thereof.

11. The method of claim 10, wherein the C₁-C₃ alcohol is ethyl alcohol.

12. The method of claim 1, wherein the animal tissue comprises human, bovine, or porcine is tissue.

13. The method of claim 1, further comprising the step of implanting the bioprosthesis in a patient.

14. The method of claim 1, wherein the chemical cross-linking agent comprises an aldehyde.

15. A method for treating a bioprosthesis comprising,
providing a bioprosthesis that comprises a fixed animal tissue that has been fixed with an aldehyde, wherein the bioprosthesis is cytotoxic upon implantation into an animal, and
contacting the bioprosthesis with an aqueous composition comprising between about 0.1 and 10% by volume of a C₇-C₉ alcohol, between about 15 and 25% by volume of a C₁-C₃ alcohol, and between about 65 and 85% by volume of an aqueous salt solution is selected from the group consisting of sodium chloride solution, triethanolamine buffer, sodium chloride solution, HEPES buffered saline, Tris buffered saline, phosphate buffered saline, HEPES buffer, Pipes buffer, Tris buffer, and Bis-Tris buffer,
whereby the bioprosthesis is less cytotoxic after being contacted with the aqueous composition than before being contacted with the aqueous composition.

16. A bioprosthesis suitable for implantation into an animal prepared by a method comprising,
providing a bioprosthesis that comprises an animal tissue that has been fixed with a chemical cross-linking agent, wherein the bioprosthesis is cytotoxic upon implantation into an animal, and
contacting the bioprosthesis with an aqueous composition comprising at least one salt and a C₁-C₃ alcohol, whereby the bioprosthesis is less cytotoxic after being contacted with the aqueous composition than before being contacted with the aqueous composition.

17. A method for treating an implantable medical device comprising,
providing an implantable medical device that comprises at least one viral contaminant, and
contacting the implantable medical device with an aqueous composition comprising at least one salt, a C₄-C₃₆ alcohol, and a C₁-C₃ alcohol, whereby the implantable medical device comprises less viral contaminant after being contacted with the aqueous composition than before being contacted with the aqueous composition.

18. The method of claim 17, wherein the C₄-C₃₆ alcohol is a C₆-C₁₈ alcohol.

19. The method of claim 18, wherein the C₆-C₁₈ alcohol is selected from the group consisting of heptanol, octanol, nonanol, 1,2-octanediol, 1,8-octanediol, 1,10-decanol, 1,10-dodecanol, 1,2-dihydroxydecane, 1,2-dihydroxydodecane, and mixtures thereof.

20. The method of claim 19, wherein the C₆-C₁₈ alcohol is 1,2-octanediol.

21. The method of claim 18, wherein the aqueous composition comprises between about 0.1 and 10% by volume of the C₆-C₁₈ alcohol, between about 15 and 45% by volume of the C₁-C₃ alcohol, and between about 45 and 85% by volume of an aqueous salt solution.

22. The method of claim 21, wherein the aqueous salt solution comprises a chemical buffer.

23. The method of claim 22, wherein the aqueous salt solution is selected from the group consisting of triethanolamine buffer, HEPES buffered saline, Tris buffered saline, phosphate buffered saline, HEPES buffer, PIPES buffer, Tris buffer, and Bis-Tris buffer.

24. The method of claim 23, wherein the aqueous salt solution is HEPES buffer.

25. The method of claim 17, wherein the C₁-C₃ alcohol is selected from the group consisting of isopropyl alcohol, ethyl alcohol, methyl alcohol, and mixtures thereof.

26. The method of claim 25, wherein the C₁-C₃ alcohol is ethyl alcohol.

27. The method of claim 17, wherein the implantable medical device comprises an animal tissue.

28. The method of claim 27, wherein the animal tissue comprises human, bovine, or porcine tissue.

29. The method of claim 28, wherein the animal tissue is fixed.

30. The method of claim 29, wherein the animal tissue is fixed with a chemical cross-linking agent.

31. The method of claim 30, wherein the chemical cross-linking agent comprises an aldehyde.

32. The method of claim 17, further comprising the step of implanting the bioprosthesis in a patient.

33. The method of claim 17, wherein the amount of viral contaminant is reduced by at least about one log by contacting the implantable medical device with the aqueous composition.

34. The method of claim 33, wherein the amount of viral contaminant is reduced by between about one and five logs by contacting the implantable medical device with the aqueous composition.

35. The method of claim 17, wherein the implantable medical device is treated with at least one antiviral treatment before being contacted with the aqueous composition.

36. The method of claim 35, wherein the at least one antiviral treatment comprises contacting the implantable medical device with an aldehyde.

37. The method of claim 35, wherein the implantable medical device comprises an animal tissue.

38. An medical device suitable for implantation into an animal prepared by a method comprising,
providing an implantable medical device that comprises at least one viral contaminant, and
contacting the implantable medical device with an aqueous composition comprising at least one salt, a C₄-C₃₆ alcohol, and a C₁-C₃ alcohol, whereby the implantable medical device comprises less viral contaminant after being contacted with the aqueous composition than before being contacted with the aqueous composition.

39. The method of claim 35, wherein the implantable medical device is treated with at least one antiviral treatment before being contacted with the aqueous composition.

40. A method for treating an implantable medical device comprising,
providing an implantable medical device that comprises at least one viral contaminant, and
contacting the implantable medical device with a aqueous composition comprising about 0.1 and 10% by volume of a C₇-C₉ alcohol, between about 15 and 45% by volume of a C₁-C₃ alcohol, and between about 45 and 85% by volume of an aqueous salt solution selected from the group consisting of sodium chloride solution, triethanolamine buffer, sodium chloride solution, HEPES buffered saline, Tris buffered saline, phosphate buffered saline, HEPES buffer, Pipes buffer, Tris buffer, and Bis-Tris buffer,
whereby the implantable medical device comprises at least about one log less viral contaminant after being contacted with the aqueous composition than before being contacted with the aqueous composition.

41. The method of claim 36, whereby the implantable medical device comprises between about one and five logs less viral contaminant after being contacted with the aqueous composition than before being contacted with the aqueous composition.

42. The method of claim 36, wherein the implantable medical device is treated with at least one antiviral treatment before being contacted with the aqueous composition.
